(19) United States
(12) Patent Application Publication
(10) Pub. No.: US 2012/0211446 A1
(43) Pub. Date: Aug. 23, 2012

(54) MEDICAL EQUIPMENT SHELF SYSTEM AND METHOD

(76) Inventor: Jocelyn Côté, St-Hubert (CA)

(21) Appl. No.: 13/334,497

(22) Filed: Dec. 22, 2011

Related U.S. Application Data
(60) Provisional application No. 61/426,206, filed on Dec. 22, 2010.

(51) Int. Cl. A61B 19/00; B23P 11/00

(52) U.S. Cl. 211/1.57; 211/85.13; 29/428

(57) ABSTRACT

The medical equipment shelf system is for use with a suspension system. The system includes a support plate having a substantially horizontal upper surface receiving the medical equipment thereon. The support plate includes a bottom hub for engagement by a wheeled transport unit. A base plate removably receives and positions the support plate thereon. The base plate is coupled to the suspension system. A method of removably coupling a medical equipment to a suspension system is also disclosed.

### CROSS-REFERENCE TO RELATED APPLICATION

The present patent application claims the benefit of U.S. Provisional Patent Application No. 61/426,206, filed on Dec. 22, 2010, and incorporated herein by reference.

### TECHNICAL FIELD

The present invention relates to medical equipment shelf systems and methods.

### BACKGROUND

Some medical equipment are too heavy and cumbersome to be carried easily by hand. This equipment is thus generally mounted on wheeled carts or the like. However, there is often a need to mount medical equipment on a suspension system attached to the ceiling or to a wall. Transferring the medical equipment in and out of such suspension system, however, can be challenging. For instance, ventilators used in operating rooms or the like regularly need to be moved out of the room for maintenance. A defective ventilator may also need to be replaced on a very short notice. In practice, this can require extensive labor and time. Furthermore, mounting new equipment replacing older machines can require that the suspension system be replaced or modified, thereby increasing time, costs and complexity of such task.

Clearly, room for improvements exists in this area of technology.

### SUMMARY

In one aspect, there is provided a medical equipment shelf system for use with a suspension system, the medical equipment shelf system including: a support plate having a substantially horizontal upper surface receiving the medical equipment thereon, the support plate including a bottom hub for engagement by a wheeled transport unit; and a base plate removably receiving and positioning the support plate thereon, the base plate being coupled to the suspension system.

In another aspect, there is provided a method of removably coupling a medical equipment to a suspension system, the method including: mounting the medical equipment on a support plate to form a subassembly; transporting the subassembly using a wheeled transport unit; positioning the subassembly over a base plate; aligning the support plate with reference to the base plate; lowering the subassembly until the support plate is supported by the base plate; and then moving the wheeled transport unit away from the subassembly.

Further details on these aspects as well as other aspects of the proposed concept will be apparent from the following detailed description and the appended figures.

### BRIEF DESCRIPTION OF THE FIGURES

FIG. 1 is an isometric view illustrating an example of a medical equipment shelf system incorporating the proposed concept;

FIG. 2 is an isometric view illustrating the upper face of the support plate of the system shown in FIG. 1;

FIG. 3 is an isometric view illustrating the bottom face of the support plate of the system shown in FIG. 1;

FIG. 4 is a view similar to FIG. 1 but showing the system in an extended position without the support plate;

FIG. 5 is an isometric view illustrating an example of a wheeled transport unit that can be used with the system shown in FIG. 1;

FIG. 6 is a view similar to FIG. 5 but showing the wheeled transport unit in a raised position;

FIG. 7 is an enlarged isometric view illustrating the telescopic arm units of the system shown in FIG. 1;

FIG. 8 is an isometric view illustrating the locking mechanism of one of the telescopic arm units of the system shown in FIG. 1;

FIG. 9 is a view similar to FIG. 8 but showing the locking mechanism when the push button is depressed; and

FIG. 10 is a view similar to FIG. 1 but showing the rear of the system without the casing for the lifting mechanism.

### DETAILED DESCRIPTION

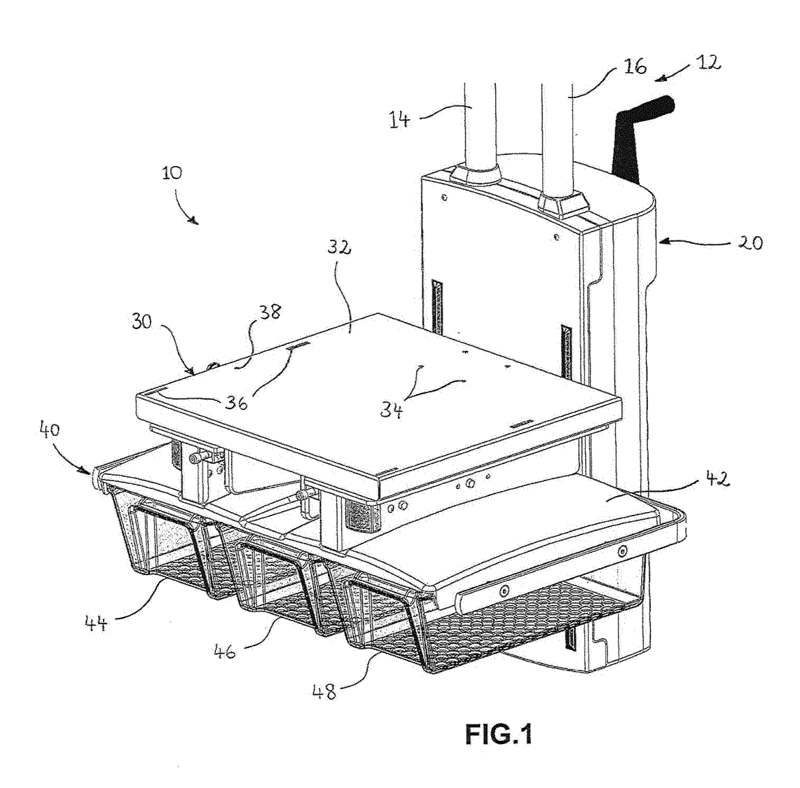

FIG. 1 is an isometric view illustrating an example of a medical equipment shelf system 10 incorporating the proposed concept. The system 10 is mounted on a suspension system 12, for instance a suspension system 12 attached to the ceiling or to a wall of an operating room or the like. The system 10 is attached to two spaced-apart vertical poles 14, 16 extending from the bottom of an overhead portion (not shown) of the suspension system 12. The system 10 as illustrated also includes a lifting unit 20 by which the other components of the system 10 can coupled to the suspension system 12. Further details on the lifting unit 20 will be given later in the text.

The system 10 includes a removable support plate 30 providing a substantially flat horizontal surface 32 on its upper face. This upper surface 32 is where the medical equipment is mounted, for instance a ventilator. It should be noted that although the present detailed description will now refer to a ventilator as the medical equipment, the proposed concept is not limited to ventilators. Other kinds of medical equipment can be used as well.

If desired, it is possible to mount each one among a plurality of ventilators on a corresponding support plate and to use the ventilators interchangeably as needed.

The ventilator can be rigidly connected to the support plate 30. For instance, as shown, the support plate 30 includes threaded holes 34 and a plurality of slots 36 near the edges thereof. These slots 36 can receive a corresponding member downwardly projecting from the ventilator. A hand-operated catch mechanism located under hole 38 can also be provided on the side of the support plate 30 as an additional safety feature. Other arrangements and configurations are possible as well.

FIG. 1 also shows a medical toolbox 40 provided under the system 10. This medical toolbox 40 includes an upper wall 42 to which are suspended three drawers 44, 46, 48. These drawers 44, 46, 48 can be very useful for storing medical supplies or other items that must be on hand. Nevertheless, omitting this medical toolbox 40 or using a different one is also possible.

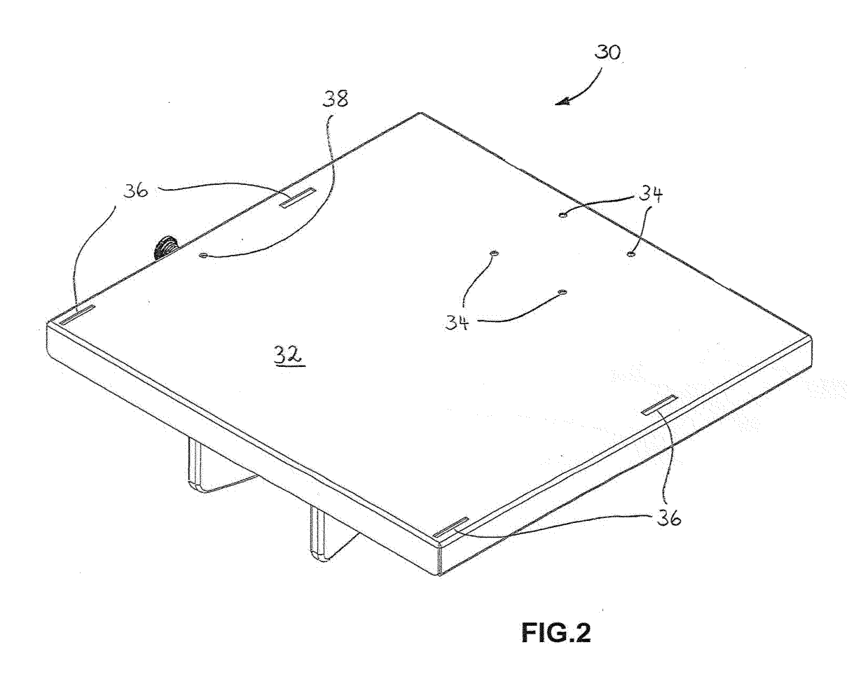

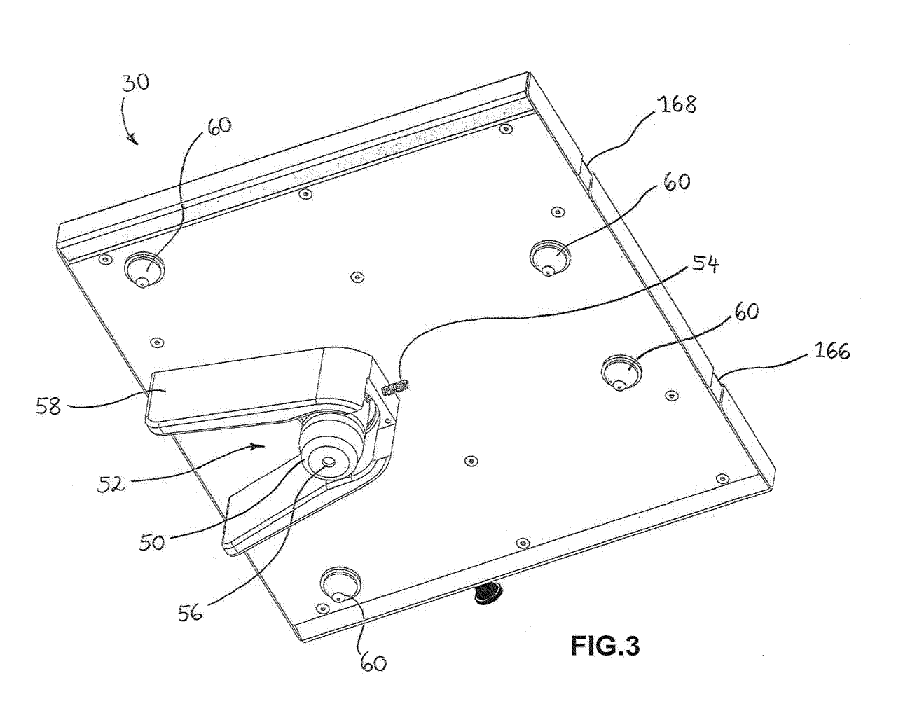

FIG. 2 is an isometric view illustrating the upper face of the support plate 30 of the system 10 shown in FIG. 1. FIG. 3 is an isometric view illustrating the bottom face of the support plate 30 of the system 10 shown in FIG. 1.

As can be seen in FIG. 3, the bottom face of the support plate 30 includes a hub 50 projecting vertically therefrom. The bottom hub 50 can have a generally circular cross section. The hub 50 is substantially in registry with the center of gravity of the subassembly formed by the support plate 30 and the ventilator mounted thereon. It does not necessarily correspond to the geometrical center of the support plate 30, as shown.

The hub 50 includes an internal spring-loaded mechanism 52 controlling a latch pin 54 projecting on the side thereof. The internal spring-loaded mechanism 52 is actuated through a center hole 56 provided on the bottom face of the hub 50. Inserting a rod-like clement through the center hole 56 retracts the latch pin 54. The support plate 30 also includes a somewhat V-shaped guide 58 and a set of four spaced-apart shallow guiding pegs 60. More details on these components will follow.

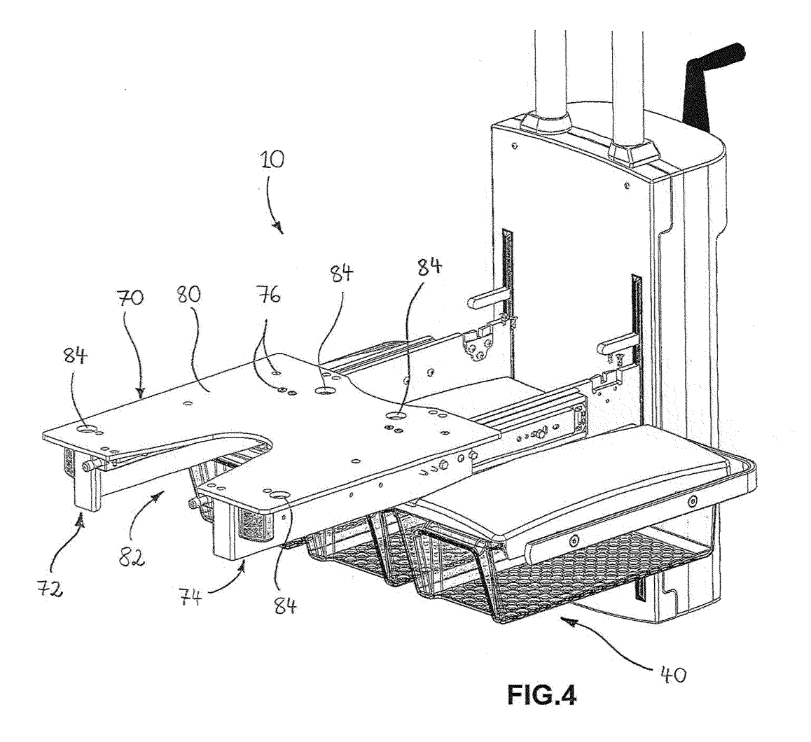

FIG. 4 is a view similar to FIG. 1 but showing the system 10 in an extended position without the support plate 30. FIG. 4 shows that the system 10 further includes a base plate 70 that is rigidly connected to two telescopic arm units 72, 74 using a plurality of screws 76. The base plate 70 is configured and disposed to receive the support plate 30 thereon. Each telescopic arm unit 72, 74 includes a movable rail and a fixed rail. The movable rail is slidingly coupled to the fixed rail, for instance using rollers, linear bearings or other arrangements.

The base plate 70 includes a flat upper surface 80. The base plate 70 also includes a large opening 82 on the front side. This opening 82 receives the substantially V-shaped guide 58 provided under the support plate 30. There are also four spaced-apart holes 84 on the base plate 70, each corresponding to one of the guiding pegs 60. These holes 84 receive the guiding pegs 60 of the support plate 30 when it is properly positioned over the base plate 70.

At the same time, the edge of the opening 82 on the front side will be engaged by the substantially V-shaped guide 58. The latch pin 54, when it is in an extended position, engages the bottom surface of the base plate 70 and locks the support plate 30 thereon.

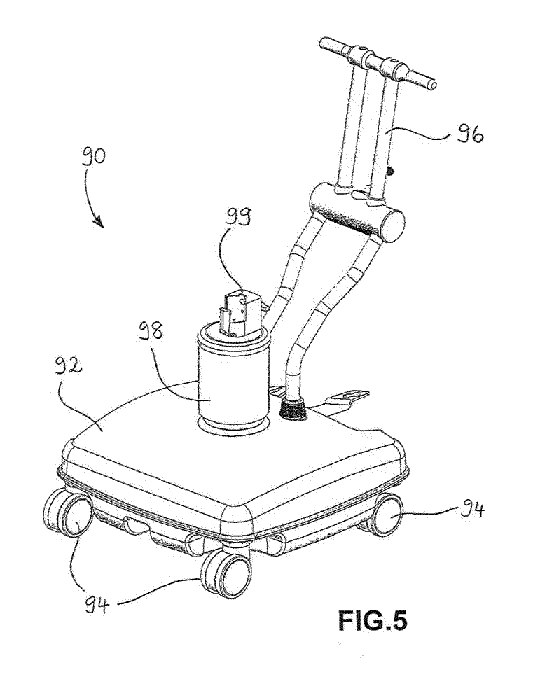

Since the subassembly formed by the ventilator and the support plate 30 can be very heavy, a wheeled transport unit can be used to conveniently move the subassembly to and away from the base plate 70. FIG. 5 is an isometric view illustrating an example of a wheeled transport unit 90 that can be used with the system 10 shown in FIG. 1. This wheeled transport unit 90 includes a base 92 supported by a plurality of wheels 94 and that can be handled using a handlebar 96. If desired, the wheeled transport unit 90 can be self-propelled using an electric motor or the like. It further includes a telescopic vertical lifting post 98 whose upper head 99 can be selectively brought into engagement with the bottom hub 50 of the support plate 30.

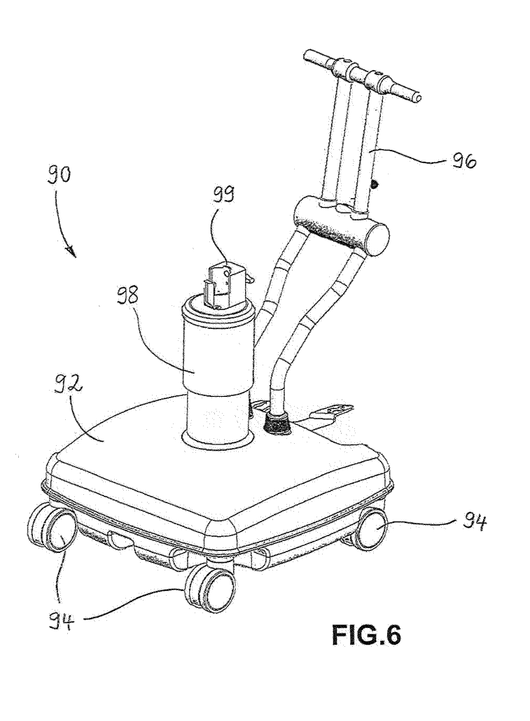

FIG. 6 is a view similar to FIG. 5 but showing the wheeled transport unit 90 in a raised position. This position allows the wheel transport unit 90 to positively engage the hub 50 under the support plate 30 when the system 10 is in an extended position, as shown in FIG. 4. As the support plate 30 and the ventilator thereon are lifted, an upwardly-projecting peg provided on the upper head 99 of the lifting post 98 will push on the internal spring-loaded mechanism 52 inside the hub 50 to retract the latch pin 54, thereby unlocking the support plate 30 from the base plate 70. Lifting the lifting post 98 furthermore will allow the guiding pegs 60 to be fully moved out of their corresponding holes 84. It will then be possible to move the wheeled transport unit 90 backward until the support plate 30 is away from the base plate 70.

To put the support plate 30 (or another support plate) back on the base plate 70, the same steps are performed in the reverse order. The substantially V-shaped guide 58 will then help the user handling the wheeled transport unit 90 to position the support plate 30 at the right location before it can be lowered.

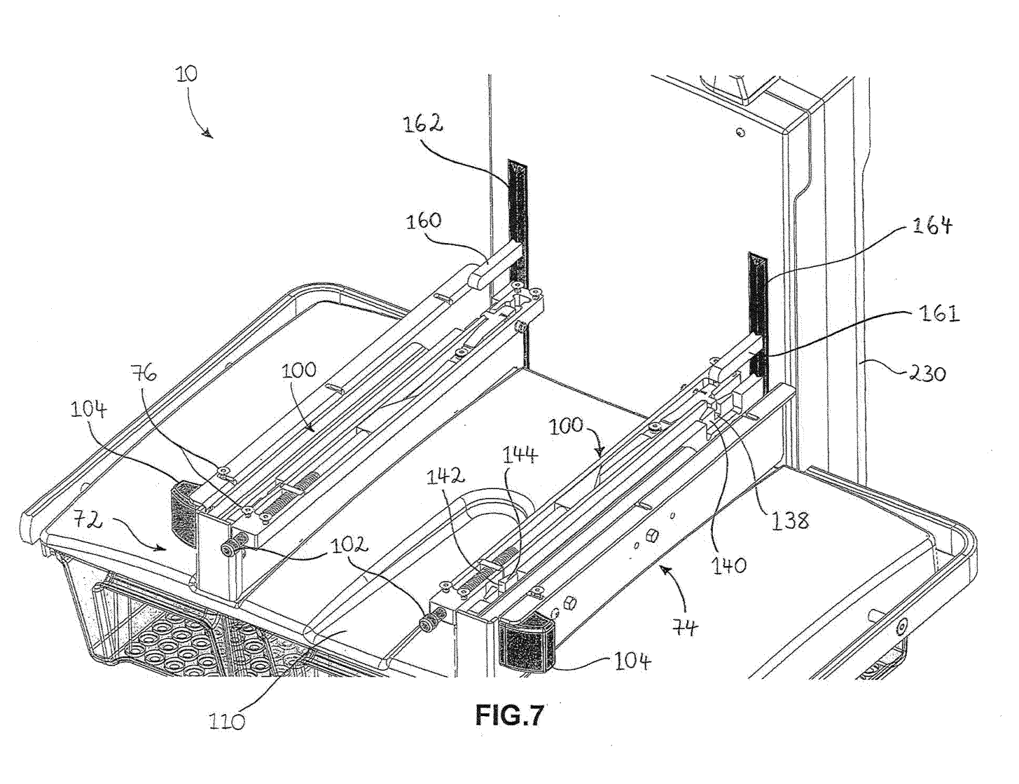

FIG. 7 is an enlarged isometric view illustrating the telescopic arm unit 72, 74 of the system 10 shown in FIG. 1. The base plate 70 is thus not illustrated in FIG. 7. However, the various screws 76 provided to attach the base plate 70 on the telescopic arm units 72, 74 are shown. This view shows locking mechanisms 100 mounted on the movable rail of the telescopic arm units 72, 74. Both telescopic arm units 72, 74 have the same kind of locking mechanism 100 but one is the mirror image of the other. Each locking mechanism 100 is designed to automatically lock the corresponding telescopic arm units 72, 74 when the base plate 70 reaches either the extended position or the retracted position. Each locking mechanism 100 further includes a push button 102 horizontally projecting on the front side of the system 10. These push buttons 102 are designed to be depressed by a user using the thumbs. Pressing on both push buttons 102 simultaneously can release the locking engagement and allow the user to slide the base plate 70. A lateral finger pad 104 is provided to each external side of the telescopic rail units 72, 74 to facilitate the handling by the users.

It should be noted that the system 10 could be used without telescopic arm units 72, 74, especially if no medical toolbox 40 is present under the base plate 70.

As best shown in FIG. 7, the upper wall 42 of the medical toolbox 40 includes a grooved portion 110 to accommodate the hub 50 under the support plate 30 when the base plate 70 is in a retracted position.

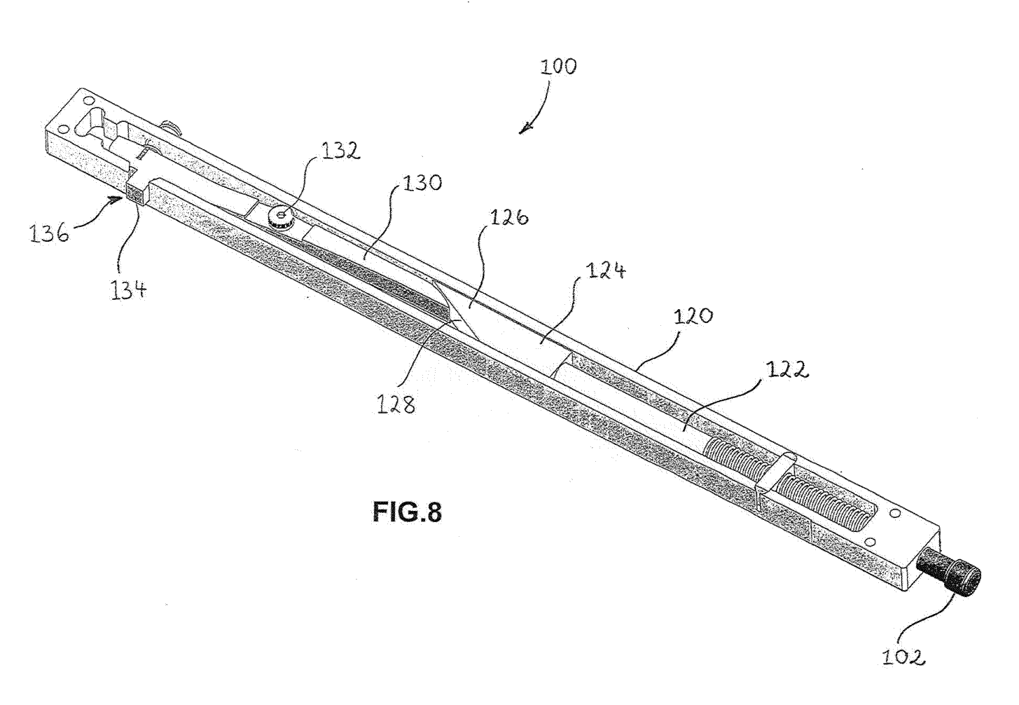

FIG. 8 is an isometric view illustrating the locking mechanism 100 of one of the telescopic arm units 72, 74 of the system 10 shown in FIG. 1. The locking mechanism 100 is in a locked position in FIG. 8. As can be seen, the locking mechanism 100 includes an elongated slotted member 120. The push button 102 extends out at one end. Pushing on the push button 102 moves a shaft 122 inside the slotted member 120 in the same direction. The distal end of the shaft 122 is connected to a sliding block 124 having a wedge-shaped end 126. Its oblique surface 128 engages the mating end of a pivot member 130. The pivot member 130 pivots around an axle 132. The pivot member 130 includes a lateral projection 134 extending out of an aperture 136 of the slotted member 120. As best shown in FIG. 7, the lateral projection 134 also engages the edges of a lateral slot 138 provided on a first member 140 connected to the corresponding fixed rail. The lateral projection 134 thus acts as a deadbolt. In the extended position, it will engage the edges of the lateral slot 142 of a second member 144 that is also connected to the same fixed rail.

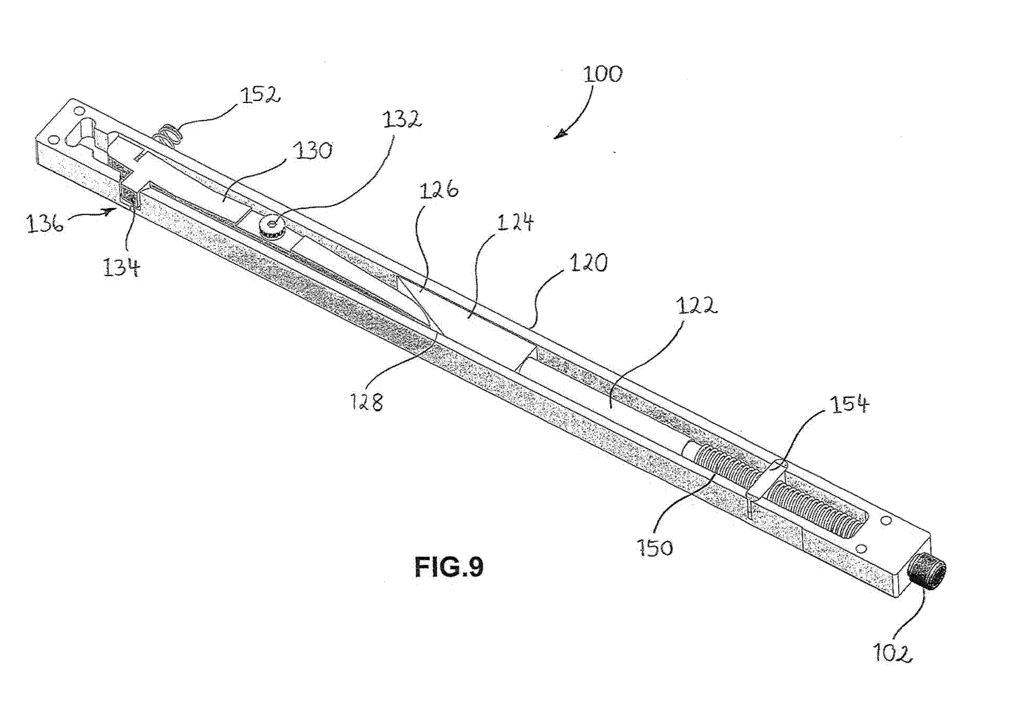

FIG. 9 is a view similar to FIG. 8 but showing the locking mechanism 100 when the push button 102 is depressed. The locking mechanism 100 is thus in an unlocked position in FIG. 9. A first return spring 150 coaxially mounted around the backside of the push button 102 and a second return spring 152 engaging the backside of the pivot member 130 bias the locking mechanism 100 into the locked position unless someone presses the push button 102. The return force of the first return spring 150 can be adjusted using a U-shaped plate 154 inserted in a corresponding seat.

As can be seen in FIG. 7, the illustrated example includes two horizontal members 160, 161 projecting from the slots 162, 164 of the lifting unit 20. These horizontal members 160 are configured and disposed to engage a corresponding one of apertures 166, 168 (FIG. 3) provided on the rear edge of the support plate 30 when the base plate 70 is in a retracted position. This can be used as an additional safety measure to prevent the support plate 30 from moving.

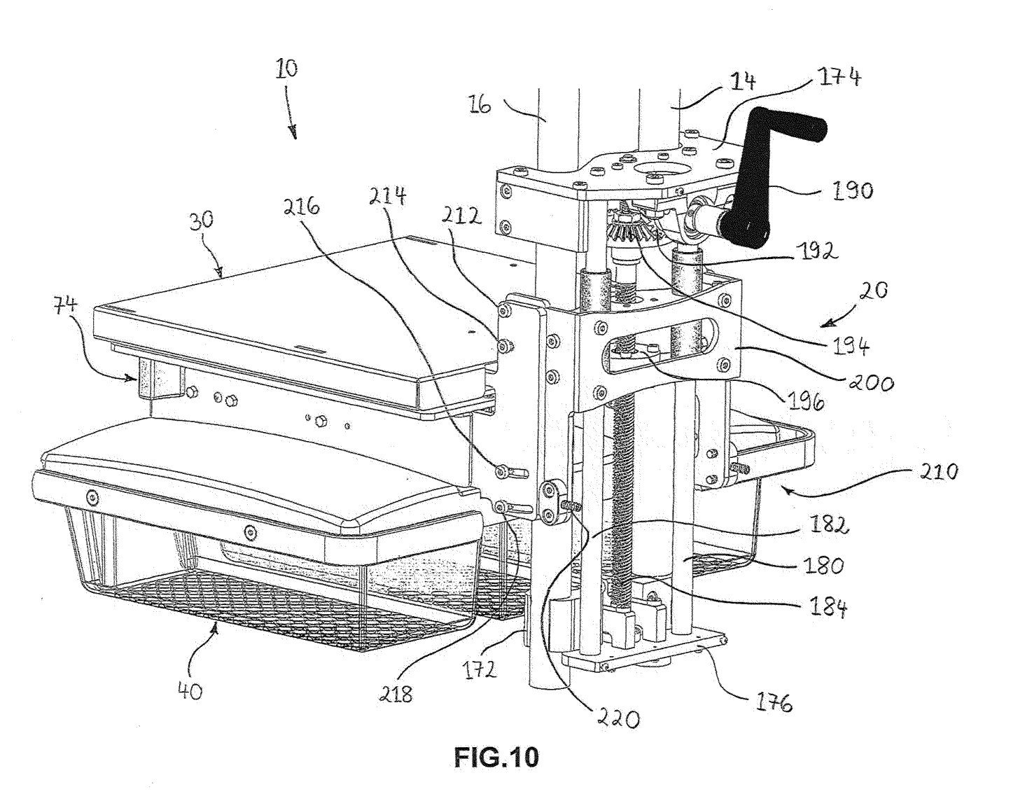

FIG. 10 is a view similar to FIG. 1 but showing the rear of the system 10 without the casing 230 for the lifting mechanism 20. The casing 230 is visible in FIG. 7 for instance. FIG. 10 shows the various clamps 170, 172 engaging the two vertical poles 14, 16. These clamps 170, 172 support an upper portion 174 of the lifting unit 20 and a bottom portion 176 thereof. Two vertical guide posts 180, 182 and an elongated vertical screw 184 extend between the upper and bottom portions 174, 176. The screw 184 in the illustrated example can be rotated using a hand-operated crank handle 190 pivotally mounted to the upper portion 174 of the lifting mechanism 20. The crank handle 190 rotates a first gear 192 in mesh with a second gear 194 coaxially mounted on the screw 184. In use, rotating the crank handle 190 will rotate the screw 184 on itself. This will move the threaded follower 196 of a movable frame 200 up or down, depending on the direction of the rotation. The pitch of the screw 184 and of the follower 194 is chosen to prevent the screw 184 from moving by itself under the weight of the support plate 30 and the ventilator mounted thereon.

The movable frame 200 is slidably connected to the vertical guide posts 180, 182. This way, rotating the crank handle 190 will move the movable frame 200 up or down. Other configurations and arrangements are possible as well. For instance, one can use an electric motor or the like to rotate the screw. Other variants are possible.

Also in the illustrated example, the base of the telescopic arm units 72, 74 is connected to the movable frame 200 through an adjustable interface 210. The interface 210 allows the angle of the telescopic arm units 72, 74 to be adjusted so as to compensate for the weight of the ventilator. This adjustment, however, is intended to be a factory setting. On each side, the base of the telescopic arm unit 72, 74 can pivot, with reference to the corresponding side portion of the movable frame 200, around the bolt 212 at the top. Oblong holes are provided in the base of the telescopic arm units 72, 74 for the three other bolts 214, 216, 218. To adjust the angle, the tension in all bolts 212, 214, 216, 218 is released and the angle is set by rotating the set screws 220 located at the back. The front end of each set screw 220 pushes on the rear bottom edge of the base of the telescopic arm unit 72, 74. Rotating these set screws 220 will thus modify the angle. Once the angle is set, the side bolts 212, 214, 216, 218 can be tightened.

Overall, the proposed concept provides a method of removably coupling a medical equipment to a suspension system 12. The method includes:
mounting the medical equipment on a support plate 30 to form a subassembly;
transporting the subassembly using a wheeled transport unit 90;
positioning the subassembly over a base plate 70;
aligning the support plate 30 with reference to the base plate 70;
lowering the subassembly until the support plate 30 is supported by the base plate 70; and then
moving the wheeled transport unit 90 away from the subassembly.

The present detailed description and the appended figures are meant to be exemplary only, and a skilled person will recognize that variants can be made in light of a review of the present disclosure without departing from the proposed concept.

What is claimed is:

1. A medical equipment shelf system for use with a suspension system, the medical equipment shelf system including:
a support plate having a substantially horizontal upper surface receiving the medical equipment thereon, the support plate including a bottom hub for engagement by a wheeled transport unit; and
a base plate removably receiving and positioning the support plate thereon, the base plate being coupled to the suspension system.

2. The system as defined in claim 1, further including a lifting unit positioned between the base plate and the suspension system, the lifting unit being configured and disposed to vary the base plate in height.

3. The system as defined in claim 2, wherein the lifting unit includes a vertically-disposed screw in mesh with a threaded follower, the screw being rotated to change a relative position of the follower with reference to the screw and thereby vary the base plate in height.

4. The system as defined in claim 3, wherein the screw is rotated through a gear set.

5. The system as defined in claim 4, wherein the screw is rotated either by a hand-operated crank or by an electrical motor.

6. The system as defined in claim 1, wherein the base plate is mounted to a pair of telescopic arm units, the base plate being selectively movable in a horizontal plate between a retracted position and an extended position.

7. The system as defined in claim 6, wherein each telescopic arm unit includes a locking mechanism to lock the base plate in the retracted position and in the extended position.

8. The system as defined in claim 7, wherein the locking mechanism is automatically engaged when the base plate reaches either the retracted position or the extended position.

9. The system as defined in claim 8, wherein the locking mechanism includes a two-stage mechanism operated by a push button.

10. The system as defined in claim 1, further including an angle-adjustment mechanism, the angle-adjustment mechanism compensating for a change in the angle relative to the horizontal when the medical equipment is installed in the suspension system.

11. The system as defined in claim 1, further including a medical toolbox provided under the base plate.

12. A method of removably coupling a medical equipment to a suspension system, the method including:
mounting the medical equipment on a support plate to form a subassembly;
transporting the subassembly using a wheeled transport unit;
positioning the subassembly over a base plate;
aligning the support plate with reference to the base plate;
lowering the subassembly until the support plate is supported by the base plate; and then
moving the wheeled transport unit away from the subassembly.

13. The method as defined in claim 12, wherein lowering the subassembly until the support plate is supported by the base plate includes locking the support plate to the base plate.

14. The method as defined in claim 12, further including the subsequent step of:
moving the base plate with the subassembly from an extended position to a retracted position.

15. The method as defined in claim 14, wherein moving the base plate with the subassembly from the extended position to the retracted position includes releasing a locking mechanism by pressing a push button.
